(19) United States
(12) Patent Application Publication
(10) Pub. No.: US 2019/0144273 A1
(43) Pub. Date: May 16, 2019

(54) PROCESS FOR PROVIDING HEAT TO INDUSTRIAL FACILITIES

(71) Applicant: ExxonMobil Research and Engineering Company, Annandale, NJ (US)

(72) Inventor: Mohsen N. Harandi, New Hope, PA (US)

(21) Appl. No.: 16/190,200

(22) Filed: Nov. 14, 2018

Related U.S. Application Data
(60) Provisional application No. 62/587,102, filed on Nov. 16, 2017.

(51) Int. Cl. C01B 3/38; C01B 3/36; C07C 29/151; C10L 1/04; C07C 1/04; C06B 43/00; F23C 10/22

(52) U.S. Cl. CPC C01B 3/382; C10L 2270/023; C07C 29/1518; C10L 1/04; C07C 1/0485; C06B 43/00; F23C 10/22; C01B 2203/025; C01B 2203/0244; C01B 2203/0495; C01B 2203/0475; C01B 2203/061; C01B 2203/062; C10L 2200/0423; C01B 3/36

(57) ABSTRACT

A process for providing heat to an industrial facility comprises contacting a hydrocarbon fuel with oxygen in a reaction zone under partial oxidation conditions including a below stoichiometric oxygen to fuel molar ratio for full combustion to generate heat in the reaction zone and produce a gaseous effluent stream containing carbon monoxide. At least part of the carbon monoxide from the gaseous effluent stream is converted to one or more of chemical products different from carbon monoxide transferring at least part of the heat generated in reaction zone and/or contained in the gaseous effluent stream is transferred to a separate operation in the industrial facility.

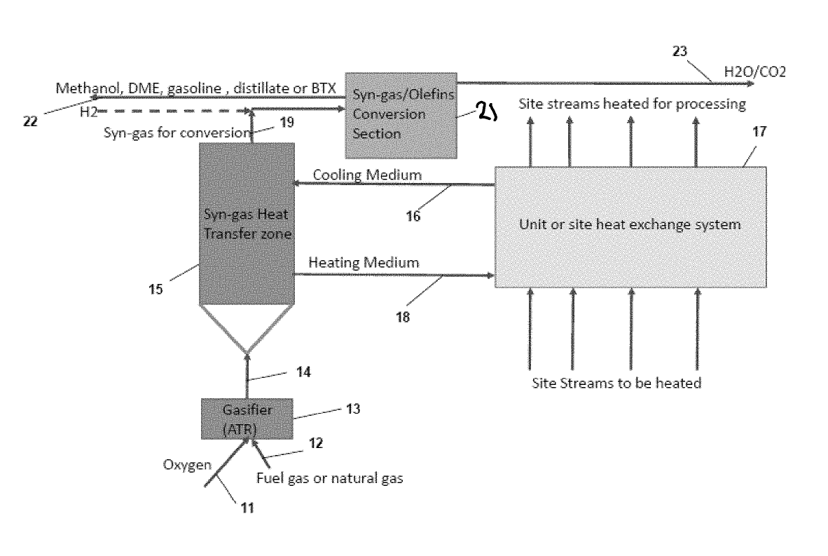

### CROSS-REFERENCE TO RELATED APPLICATIONS

This application claims priority to U.S. Provisional Application Ser. No. 62/587,102 filed Nov. 16, 2017, which is herein incorporated by reference in its entirety.

### FIELD

The present disclosure relates to a process for providing heat to industrial facilities, particularly refineries, chemical and petrochemical plants.

### BACKGROUND

For decades industrial facilities have burned hydrocarbon fuels to provide the heat necessary to run their major processes. Currently this involves the long practiced reaction of the complete combustion of the fuel with air in boilers and fired heaters. However, this not only results in fuel usage inefficiency for heating up and processing the nitrogen in the air and emitting flue gas at a temperature typically at about 400-500° F. but also leads to significant emission of undesirable compounds, such as CO₂, butadiene, NOₓ and SOₓ. Separation of all these pollutants from the flue gas containing mainly N₂ at low pressure is technically and economically challenging.

There is therefore significant value in the development of alternative processes for providing heat to industrial facilities.

It is known that fuels including methane, the major constituent of natural gas, undergo partial oxidation or gasification in the absence of a catalyst and under controlled oxygen conditions to produce carbon monoxide either alone or in the presence of hydrogen, as synthesis gas. The partial oxidation reaction is exothermic and is normally conducted at an outlet temperature of at least 1500° F. (815° C.) and a pressure greater than 20 psig (239 kPa-a), more generally 100 to 1000 psig (790 to 7000 kPa-a). Depending on the fuel used and products to be made the H₂ to CO ratio of the synthesis gas product can be controlled by addition of steam for conducting the water gas shift reaction and/or by the addition of H₂.

It is also known that synthesis gas can be converted to valuable chemicals, including gasoline and distillate, by the Fischer-Tropsch process and via methanol synthesis processes. In addition, synthesis gas can be converted to dimethyl ether and many other oxygenates and hydrocarbons. These processes are typically operated at temperatures from 40 to 600° C.

### SUMMARY

Analysis of conventional industrial facilities, particularly refineries and petrochemical plants, shows that most processes within these facilities operate at a temperature of 980° F. (527° C.) or below, which is in excess of 500° F. (277° C.) below the temperature of the effluent generated in the gasification of methane. Thus gasification of methane and other fuels provides a potential source of heat for these operations, while offering the advantages of reduced harmful emissions as compared with conventional fuel combustion processes and the production of carbon monoxide as an additional source of valuable chemicals. Also disclosed herein is novel system of transferring heat from combustion gases involving a fluid bed of refractory and/or catalytic material.

Accordingly, in one aspect, the present disclosure is directed to a process for providing heat to an industrial facility, the process comprising:

(a1) contacting a hydrocarbon fuel with oxygen in a reaction zone under partial oxidation conditions including a below stoichiometric oxygen to fuel molar ratio for full combustion to generate heat in the reaction zone and produce a gaseous effluent stream containing carbon monoxide;

(b1) converting at least part of the carbon monoxide from the gaseous effluent stream to one or more of chemical products different from carbon monoxide; and

(c1) transferring at least part of the heat generated in reaction zone and/or contained in the gaseous effluent stream to an operation in the industrial facility other than the contacting (a1) and the converting (b1).

In a further aspect, the present disclosure is directed to a process for providing heat to an industrial facility, the process comprising:

(a2) contacting a hydrocarbon fuel with oxygen in a reaction zone under conditions effective to generate heat in the reaction zone and produce a gaseous effluent stream;

(b2) transferring at least part of the heat generated in reaction zone and/or contained in the gaseous effluent stream to a fluid bed comprising particles of inert, refractory material so as to directly transfer heat to the fluid bed; and then

(c2) using the fluid bed to provide heat to an operation in the industrial facility other than the contacting (a2).

### BRIEF DESCRIPTION OF THE DRAWINGS

The present disclosure is susceptible to various modifications and alternative forms, specific exemplary implementations thereof have been shown in the drawings and are herein described in detail. It should be understood, however, that the description herein of specific exemplary implementations is not intended to limit the disclosure to the particular forms disclosed herein. This disclosure is to cover all modifications and equivalents as defined by the appended claims. It should also be understood that the drawings are not necessarily to scale, emphasis instead being placed upon clearly illustrating principles of exemplary embodiments of the present invention. Moreover, certain dimensions may be exaggerated to help visually convey such principles. Further where considered appropriate, reference numerals may be repeated among the drawings to indicate corresponding or analogous elements. Moreover, two or more blocks or elements depicted as distinct or separate in the drawings may be combined into a single functional block or element. Similarly, a single block or element illustrated in the drawings may be implemented as multiple steps or by multiple elements in cooperation. The forms disclosed herein are illustrated by way of example, and not by way of limitation, the accompanying drawing and in which like reference numerals refer to similar elements and in which:

The FIGURE is a schematic diagram of a process for providing heat to an industrial facility according to one embodiment of the present disclosure.

### DETAILED DESCRIPTION OF THE EMBODIMENTS

### Terminology

The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than the broadest meaning understood by skilled artisans, such a special or clarifying definition will be expressly set forth in the specification in a definitional manner that provides the special or clarifying definition for the term or phrase.

For example, the following discussion contains a non-exhaustive list of definitions of several specific terms used in this disclosure (other terms may be defined or clarified in a definitional manner elsewhere herein). These definitions are intended to clarify the meanings of the terms used herein. It is believed that the terms are used in a manner consistent with their ordinary meaning, but the definitions are nonetheless specified here for clarity.

A/an: The articles “a” and “an” as used herein mean one or more when applied to any feature in embodiments and implementations of the present invention described in the specification and claims. The use of “a” and “an” does not limit the meaning to a single feature unless such a limit is specifically stated. The term “a” or “an” entity refers to one or more of that entity. As such, the terms “a” (or “an”), “one or more” and “at least one” can be used interchangeably herein.

About: As used herein, “about” refers to a degree of deviation based on experimental error typical for the particular property identified. The latitude provided the term “about” will depend on the specific context and particular property and can be readily discerned by those skilled in the art. The term “about” is not intended to either expand or limit the degree of equivalents which may otherwise be afforded a particular value. Further, unless otherwise stated, the term “about” shall expressly include “exactly,” consistent with the discussion below regarding ranges and numerical data.

Above/below: In the following description of the representative embodiments of the invention, directional terms, such as “above”, “below”, “upper”, “lower”, etc., are used for convenience in referring to the accompanying drawing. In general, “above”, “upper”, “upward” and similar terms refer to a direction toward the earth's surface along a wellbore, and “below”, “lower”, “downward” and similar terms refer to a direction away from the earth's surface along the wellbore. Continuing with the example of relative directions in a wellbore, “upper” and “lower” may also refer to relative positions along the longitudinal dimension of a wellbore rather than relative to the surface, such as in describing both vertical and horizontal wells.

And/or: The term “and/or” placed between a first entity and a second entity means one of (1) the first entity, (2) the second entity, and (3) the first entity and the second entity. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements). As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of”.

Any: The adjective “any” means one, some, or all indiscriminately of whatever quantity.

At least: As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements). The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

Based on: “Based on” does not mean “based only on”, unless expressly specified otherwise. In other words, the phrase “based on” describes both “based only on,” “based at least on,” and “based at least in part on.”

Comprising: In the claims, as well as in the specification, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Determining: “Determining” encompasses a wide variety of actions and therefore “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

Embodiments: Reference throughout the specification to “one embodiment,” “an embodiment,” “some embodiments,” “one aspect,” “an aspect,” “some aspects,” “some implementations,” “one implementation,” “an implementation,” or similar construction means that a particular component, feature, structure, method, or characteristic described in connection with the embodiment, aspect, or implementation is included in at least one embodiment and/or implementation of the claimed subject matter. Thus, the appearance of the phrases “in one embodiment” or “in an embodiment” or “in some embodiments” (or “aspects” or “implementations”) in various places throughout the specification are not necessarily all referring to the same embodiment and/or implementation. Furthermore, the particular features, structures, methods, or characteristics may be combined in any suitable manner in one or more embodiments or implementations.

Exemplary: “Exemplary” is used exclusively herein to mean “serving as an example, instance, or illustration.” Any embodiment described herein as “exemplary” is not necessarily to be construed as preferred or advantageous over other embodiments.

May: Note that the word “may” is used throughout this application in a permissive sense (i.e., having the potential to, being able to), not a mandatory sense (i.e., must).

Operatively connected and/or coupled: Operatively connected and/or coupled means directly or indirectly connected for transmitting or conducting information, force, energy, or matter.

Optimizing: The terms “optimal,” “optimizing,” “optimize,” “optimality,” “optimization” (as well as derivatives and other forms of those terms and linguistically related words and phrases), as used herein, are not intended to be limiting in the sense of requiring the present invention to find the best solution or to make the best decision. Although a mathematically optimal solution may in fact arrive at the best of all mathematically available possibilities, real-world embodiments of optimization routines, methods, models, and processes may work towards such a goal without ever actually achieving perfection. Accordingly, one of ordinary skill in the art having benefit of the present disclosure will appreciate that these terms, in the context of the scope of the present invention, are more general. The terms may describe one or more of: 1) working towards a solution which may be the best available solution, a preferred solution, or a solution that offers a specific benefit within a range of constraints; 2) continually improving; 3) refining; 4) searching for a high point or a maximum for an objective; 5) processing to reduce a penalty function; 6) seeking to maximize one or more factors in light of competing and/or cooperative interests in maximizing, minimizing, or otherwise controlling one or more other factors, etc.

Order of steps: It should also be understood that, unless clearly indicated to the contrary, in any methods claimed herein that include more than one step or act, the order of the steps or acts of the method is not necessarily limited to the order in which the steps or acts of the method are recited.

Ranges: Concentrations, dimensions, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a range of about 1 to about 200 should be interpreted to include not only the explicitly recited limits of 1 and about 200, but also to include individual sizes such as 2, 3, 4, etc. and sub-ranges such as 10 to 50, 20 to 100, etc. Similarly, it should be understood that when numerical ranges are provided, such ranges are to be construed as providing literal support for claim limitations that only recite the lower value of the range as well as claims limitation that only recite the upper value of the range. For example, a disclosed numerical range of 10 to 100 provides literal support for a claim reciting “greater than 10” (with no upper bounds) and a claim reciting “less than 100” (with no lower bounds).

### DESCRIPTION

Specific forms will now be described further by way of example. While the following examples demonstrate certain forms of the subject matter disclosed herein, they are not to be interpreted as limiting the scope thereof, but rather as contributing to a complete description.

Described herein is a process for providing heat to an industrial facility in which, rather than burn a hydrocarbon fuel in a conventional furnace to provide the necessary heat, the fuel is contacted with oxygen in a reaction zone under partial oxidation conditions including an oxygen to fuel molar ratio below the stoichiometric value required for full combustion to produce a gaseous effluent stream containing carbon monoxide or carbon monoxide and hydrogen, also known as synthesis gas or simply syngas. The partial oxidation reaction is highly exothermic such that heat is generated in the reaction zone and the CO-containing effluent stream leaving the partial combustion reactor is typically at a temperature of at least 1500° F. (815° C.). In the present process, at least part of the heat generated in the reaction zone and/or contained in this effluent stream is transferred to a separate downstream operation in the industrial facility, while at least part of carbon monoxide in the effluent stream is converted to other chemical compounds, such as C₂+ hydrocarbons and/or oxygenates, thereby providing an additional source of valuable chemicals to the facility. In addition, since these CO conversion processes are also generally exothermic, they can be used to provide additional, lower temperature heat to the facility. Furthermore some conversion of the methane to CO₂ may also be allowed in the partial combustion step to facilitate the heat balance.

The individual steps of the present process will now be described in more detail.

### Partial Combustion of Hydrocarbon Fuel

Any hydrocarbon-containing source material can be used as the hydrocarbon fuel for the partial combustion step of the present process. For example, the source material can comprise, e.g., methane and other lower (C₄—) alkanes, such as contained in a natural gas stream, or heavier hydrocarbonaceous materials, such as coal and biomass. Desirably, the source material comprises ≥10 vol. %, such as ≥50 vol. %, based on the volume of the source material, of at least one hydrocarbon, especially methane.

The partial oxidation is generally conducted by injecting preheated hydrocarbon, oxygen (generally as air) and optionally steam through a burner into a closed combustion chamber. Preferably, the individual components are introduced at a burner where they meet in a diffusion flame, producing oxidation products and heat. In the combustion chamber, partial oxidation of the hydrocarbons generally occurs, either in the presence or absence of a catalyst, with less than stoichiometric oxygen at very high temperatures. Preferably, the components are preheated and pressurized to reduce reaction time and enhance heat transfer. The process occurs at a temperature of at least 815° C., typically at least 900° C. and mostly at least 1000° C. and at a pressure of from atmospheric to about 150 atmosphere (15,000 kPa-a), preferably above 10 atmosphere. Where the source material contains methane, the methane is partially-oxidized to carbon monoxide, optionally together with hydrogen, according to the following representative reactions:

CH₄+½O2=CO+2H₂ (i)

CH₄+H₂O=CO+3H₂ (ii)

Competing methane and CO reactions include:

CH_{4+3/2}O₂=CO+2H₂O (iii)

CH₄+2O₂=CO₂+2H₂O (iv), and

CO+H₂O=CO₂+H₂ (v).

Thus, to minimize the competing reactions (iii) and (iv), the oxygen to methane ratio in the partial oxidation process can be below 1, preferably below 0.6. However, reaction (iii) can be desirable for heat generation while reactions (ii) and (v) are desirable for maximizing syngas H₂ content. Some extent of reaction (iv) is desirable for heat balance although at the penalty of some CO₂ production. Therefore, O₂ and steam addition ratios are controlled to obtain desired heat export per methane fuel rate while balancing the syngas composition based on the syngas conversion unit demand on preferred H₂ to CO ratio and CO₂ production tolerance. In some embodiments, the O₂ to methane molar ratio may be in the range from 0.8 to 1.3.

All or part of the combustion can take place in a fluidized bed vessel, preferably using good distribution of O₂ and fuel in various portions and levels of the bed. The fluid-bed may be operated in the bubbling or turbulent regimes to create stable bed operations while avoiding high erosion rates of tubes.

Even with the oxygen to carbon atomic ratio controlled within the above ranges, the effluent from the partial oxidation process comprises CO, CO₂, H₂ and H₂O, together with N₂ where the combustion oxygen is supplied as air and in some cases together with small quantities of unreacted methane. The CO₂, N₂ and H₂O can be removed from the effluent in known manner and treated, used and/or captured as desired.

The molar ratio of H₂:CO in the partial oxidation effluent is typically no more than 2.0, such as from about 0.1 to 2.0, which may be too low for advantageous use of the effluent in some syngas conversion process. As a result additional hydrogen may need to be introduced into the effluent. This can be achieved by supplying molecular hydrogen from an external source available to the relevant industrial facility or by adding steam to the hydrocarbon-containing source material to drive the steam reforming reaction (ii). However, since steam reforming is endothermic, it will tend to reduce the temperature of the partial oxidation effluent.

In some embodiments, it may be desirable to employ an auto-thermal reforming (ATR) reactor to effect at least part of the partial combustion step. An ATR is a form of steam reformer including a catalytic gas generator bed with a specially designed burner/mixer to which preheated hydrocarbon gas, air or oxygen, and steam are supplied. Partial combustion of the hydrocarbon in the burner supplies heat necessary for the reforming reactions that occur in the catalyst bed below the burner to form a mixture of mostly steam, hydrogen, carbon monoxide (CO), carbon dioxide (CO2), and the like.

The temperature of the effluent from the partial oxidation process will generally be reflective of the operating temperature of the process, namely at least 815° C., typically at least 900° C. and mostly at least 1000° C. Similarly, the pressure of the partial oxidation effluent will similarly be reflective of the process operating pressure and here the operating pressure can be controlled so as to provide a syngas stream at the pressure desired for optimal downstream processing. For example, if the goal is to produce distillate, it may be desirable to operate the partial oxidation process at 600 to 1000 psig (4000 to 7000 kPa-a) which provides a driving force for making heavier molecules. However, typically the pressure is set no higher than minimum available pressure for the fuel and the oxygen feed streams so as to avoid cost of compression unless higher effluent gas pressure is needed for downstream operations. Alternatively, if the goal is to make syngas that goes to a lower pressure operation, then the preference may be to operate the partial oxidation process at lower pressures, for example below 300 psig (2170 kPa-a).

Recovery of Heat from Partial Oxidation Effluent

As discussed above, most processes in industrial facilities, particularly refineries and petrochemical plants, operate at inlet temperatures of 980° F. (527° C.) or below. This applies equally to processes for converting syngas to C₂+ hydrocarbons and/or methanol. Thus, by recovering part of the heat from the partial oxidation effluent described above, the temperature of effluent can be decreased to a value consistent with conventional syngas conversion processes, thereby allowing the effluent to be a heat engine for driving processes other than syngas conversion as well as the chemical engine for driving conversion of the carbon monoxide and hydrogen in the effluent to additional valuable chemicals.

Any heat transfer system capable of operating with gas streams at temperatures in excess of 815° C. can be used to recover part of the heat from the partial oxidation effluent. For example, the heat transfer system can include a conventional tubular heat exchanger provided the metallurgy of the heat exchange tubes is adequate to deal with the hot oxidation effluent.

However, a more desirable heat transfer system may comprise a fluid-bed heat transfer arrangement, wherein at least part of the hot partial oxidation effluent is sent to a fluid bed of fluidized solids or at least a portion of the reaction is conducted in the fluidized bed wherein heat is directly transferred to the solids which can be a refractory, catalytically inert material, such as sand, or a catalyst for any desirable additional reaction. Cooling coils may also be submerged in the fluid bed. The heated solids then can be contacted with either a liquid for direct heat transfer to create a hot oil belt for exchange with various site's processing streams needing heat. Alternatively the hot solids can be used to transfer heat directly or indirectly to processing streams. Using a fluid-bed heat transfer arrangement greatly improves heat transfer efficiency as the heat transfer coefficient is at least an order of magnitude higher in a fluid bed than an industrial furnace. In addition, such an arrangement prevents exposure of heat exchange tubes to extreme and non-uniform temperatures which can cause coking and/or thermal stress issues for the tubes. In addition, the fluid-bed is relatively isothermal and can be staged for running at different temperatures in different zones of the bed. This type of system is known for use for heat transfer to fluid-bed reaction systems that contain catalyst in the fluidized bed, but is believed to be new for uniform and efficient heat transfer to non-fluid-bed processes which account for most of the heating requirements in industrial facilities. Moreover, this system can be used with both full combustion and partial combustion heat sources.

Generally, the partial oxidation effluent is supplied directly to the heat transfer system without intermediate cooling and/or separation.

### Conversion of Syngas in Partial Oxidation Effluent to Chemicals

After recovery of part of the heat from the partial oxidation effluent, typically such that the temperature of the effluent is reduced to below about 500° C., at least part of the carbon monoxide and hydrogen in the effluent stream may be converted by any known process to one or more chemical products, particularly C₂+ hydrocarbons, alcohols and ethers. The specific temperature reduction can be controlled so that the effluent stream is at the optimal temperature for the specific conversion reaction desired. Moreover, additional hydrogen can be added to the effluent stream and/or the stream can be treated to remove CO₂, N₂ and/or H₂O before the effluent is supplied to syngas conversion. In some embodiments, the N₂ and CO₂ concentrations in the effluent stream are low enough to eliminate the need for any intermediate recovery section.

One suitable syngas conversion process is the Fischer Tropsch process, which was originally developed in the 1920s and involves a series of chemical reactions that produce a variety of liquid hydrocarbons. The more useful reactions produce alkanes as follows:

(2n+1)H₂+CO→CₙH₂ₙ₊₂nH₂O

where n is typically greater than 5, such as 10-20. Most of the alkanes produced tend to be straight-chain, suitable as diesel fuel. In addition to alkane formation, competing reactions give small amounts of alkenes, as well as alcohols and other oxygenated hydrocarbons. The process is generally conducted at a temperature 150 to 300° C. in the presence of one or more catalysts, the most common of which are the transition metals cobalt, iron, and ruthenium.

In addition, a variety of methods have been developed for the production of methanol from gas mixtures containing carbon oxides and hydrogen. For example, U.S. Pat. No. 1,868,096 to Dreyfus, entitled “Manufacture of Methyl Alcohol” discloses a process for producing methanol by passing a reaction gas mixture under the requisite conditions of temperature and pressure initially over one or more catalyst masses composed of zinc oxide or zinc oxide and chromium oxide and subsequently passing said mixture over one or more methanol catalysts sensitive to sulfur poisoning such as catalysts comprising copper, manganese or compounds of copper or manganese. The methanol can then be converted to olefins and/or gasoline and distillate. For example, if the goal is to make ethylene and propylene, then it may be preferable to convert the fluid-bed cooler to a catalytic zone by replacing sand with a dual catalyst (for example chromium oxide for converting the syngas to methanol and ZSM-5 or SAPO for upgrading the methanol simultaneously to ethylene/propylene) operating at a temperature of about 540° C. and a pressure below 300 psig (2170 kPa-a)

Another suitable syngas conversion process is the multistage process for producing aromatics described in US Patent Application Publication No. 20160207846 to Soultanidis et al., entitled “Process for Converting Syngas to Aromatics and Catalyst System Suitable Therefor”, the entire contents of which are incorporated herein by reference. In this process, syngas is converted to a C₁-C₄ alcohol mixture in a first stage by contacting the syngas with a first catalyst comprising rhodium or copper at a temperature of 150 to 400° C. In a second stage, the C₁-C₄ alcohol mixture is converted into an aromatic product by contact with a second catalyst comprising a molecular sieve and at least one Group 8-14 element, the molecular sieve having a Constraint Index about 1 to 12 and a silica to alumina ratio of about 10 to 100 at effective conversion conditions, including a temperature of 250 to 600° C. The final aromatic product is rich in benzene, toluene, and xylenes (e.g. greater than 50% aromatics on a hydrocarbon basis).

It is to be noted both the reaction of partial combustion to make syngas and the conversion of syngas to heavier molecules are highly exothermic and therefore can provide heat at least at two different levels. The lower temperature level may use just conventional heat exchange since coking and high temperature stresses are no longer an issue.

One embodiment of the present process is shown in the FIGURE, in which oxygen and fuel gas are supplied through lines 11 and 12 respectively to a partial combustion reactor 13, where the fuel gas is oxidized under conditions including a below stoichiometric oxygen to fuel molar ratio for full combustion to produce an effluent gas containing carbon monoxide and hydrogen. The effluent gas is then supplied by line 14 to a heat transfer zone 15, such as a fluidized bed of particulate material.

A cooling medium, such as a cold refinery hydrocarbon stream, for example a crude oil stream, is supplied via line 16 from an on-site heat exchange system 17 to the heat transfer zone 15 to recover part of the heat from the effluent gas and produce a heating medium which is returned to the heat exchange system 17 by way of line 18. The heat exchange system can then be used to provide heat to one or more process streams in the refinery.

The cooled effluent stream leaving the heat transfer zone 15 is then fed by line 19 to a syngas conversion reactor 21, where carbon monoxide and hydrogen in the effluent stream are converted to useful chemicals. The products of the syngas conversion reactor which may include but are not limited to methanol, DME (dimethyl ether), gasoline, distillate and/or BTX (mixtures of benzene, toluene and/or the three xylene isomers, all of which are aromatic hydrocarbons) are collected in line 22 for recovery, while the steam and carbon dioxide by-products of the partial combustion process are removed via line 23.

Further illustrative, non-exclusive examples of methods according to the present disclosure are presented in the following enumerated paragraphs. It is within the scope of the present disclosure that an individual step of a method recited herein, including in the following enumerated paragraphs, may additionally or alternatively be referred to as a “step for” performing the recited action.

### ADDITIONAL EMBODIMENTS

### Embodiment 1

A process for providing heat to an industrial facility, the process comprising: (a1) contacting a hydrocarbon fuel with oxygen in a reaction zone under partial oxidation conditions including a below stoichiometric oxygen to fuel molar ratio for full combustion to generate heat in the reaction zone and produce a gaseous effluent stream containing carbon monoxide; (b1) converting at least part of the carbon monoxide from the gaseous effluent stream to one or more of chemical products different from carbon monoxide; and (c1) transferring at least part of the heat generated in reaction zone and/or contained in the gaseous effluent stream to an operation in the industrial facility other than the contacting (a1) and the converting (b1).

### Embodiment 2

The process of Embodiment 1, wherein steam is also supplied to the reaction zone in (a1).

### Embodiment 3

The process of Embodiment 1 or Embodiment 2, wherein the reaction zone includes an auto-thermal reforming reactor.

### Embodiment 4

The process of any one of the preceding Embodiments, wherein step (c1) comprises transferring heat from at least part of the gaseous effluent stream to a fluidized bed of particulate material.

### Embodiment 5

The process of any one of the preceding Embodiments, wherein step (b1) comprises reacting at least part of the carbon monoxide in the effluent stream with hydrogen in the presence of a methanol synthesis catalyst to produce a methanol-containing product.

### Embodiment 6

The process of Embodiment 5, wherein step (b1) further comprises converting at least part of the methanol-containing product to gasoline and distillate.

### Embodiment 7

The process of any one of Embodiments 1-4, wherein step (b1) comprises contacting at least part of the carbon monoxide in the effluent stream with hydrogen in the presence of a Fischer-Tropsch catalyst under conditions effective to produce C5+ hydrocarbons.

### Embodiment 8

A process for providing heat to an industrial facility, the process comprising: (a2) contacting a hydrocarbon fuel with oxygen in a reaction zone under conditions effective to generate heat in the reaction zone and produce a gaseous effluent stream; (b2) transferring at least part of the heat generated in reaction zone and/or contained in the gaseous effluent stream to a fluid bed comprising particles of inert, refractory material so as to directly transfer heat to the fluid bed; and then (c2) using the fluid bed to provide heat to an operation in the industrial facility other than the contacting (a2).

### Embodiment 9

The process of according to Embodiment 8 and further comprising removing water and CO₂ from the effluent stream.

### Embodiment 10

The process according to anyone of the preceding embodiments, wherein the industrial facility is a refinery or a petrochemical plant.

### Embodiment 11

The process according to anyone of the preceding embodiments, wherein the gaseous effluent stream exiting the reaction zone is at a temperature of at least 1500° F. (815° C.).

### Embodiment 12

The process according to anyone of the preceding embodiments, wherein the operation in the industrial facility is conducted at an inlet temperature of 980° F. (527° C.) or below.

While the presently disclosed subject matter has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

1. A process for providing heat to an industrial facility, the process comprising:
(a1) contacting a hydrocarbon fuel with oxygen in a reaction zone under partial oxidation conditions including a below stoichiometric oxygen to fuel molar ratio for full combustion to generate heat in the reaction zone and produce a gaseous effluent stream containing carbon monoxide;
(b1) converting at least part of the carbon monoxide from the gaseous effluent stream to one or more of chemical products different from carbon monoxide; and
(c1) transferring at least part of the heat generated in reaction zone and/or contained in the gaseous effluent stream to an operation in the industrial facility other than the contacting (a1) and the converting (b1).

2. The process of claim 1, wherein steam is also supplied to the reaction zone in (a1).

3. The process of claim 2, wherein the reaction zone includes an auto-thermal reforming reactor.

4. The process of claim 1, wherein the industrial facility is a refinery or a petrochemical plant.

5. The process of claim 1, wherein the gaseous effluent stream exiting the reaction zone is at a temperature of at least 1500° F. (815° C.).

6. The process of claim 1, wherein the operation in the industrial facility is conducted at an inlet temperature of 980° F. (527° C.) or below.

7. The process of claim 1, wherein step (c1) comprises transferring heat from at least part of the gaseous effluent stream to a fluidized bed of particulate material.

8. The process of claim 1, wherein step (b1) comprises reacting at least part of the carbon monoxide in the effluent stream with hydrogen in the presence of a methanol synthesis catalyst to produce a methanol-containing product.

9. The process of claim 8, wherein step (b1) further comprises converting at least part of the methanol-containing product to gasoline and distillate.

10. The process of claim 1, wherein step (b1) comprises contacting at least part of the carbon monoxide in the effluent stream with hydrogen in the presence of a Fischer-Tropsch catalyst under conditions effective to produce C₅₊ hydrocarbons.

11. The process of claim 1 and further comprising removing water and CO₂ from the effluent stream.

12. A process for providing heat to an industrial facility, the process comprising:
(a2) contacting a hydrocarbon fuel with oxygen in a reaction zone under conditions effective to generate heat in the reaction zone and produce a gaseous effluent stream;
(b2) transferring at least part of the heat generated in reaction zone and/or contained in the gaseous effluent stream to a fluid bed comprising particles of inert, refractory material so as to directly transfer heat to the fluid bed; and then
(c2) using the fluid bed to provide heat to an operation in the industrial facility other than the contacting (a2).

13. The process of claim 12, wherein the industrial facility is a refinery or a petrochemical plant.

14. The process of claim 12, wherein the gaseous effluent stream exiting the reaction zone is at a temperature of at least 1500° F. (815° C.).

15. The process of claim 12, wherein the operation in the industrial facility is conducted at an inlet temperature of 980° F. (527° C.) or below.

16. The process of claim 12 and further comprising removing water and CO₂ from the effluent stream.
